(19) United States
(12) Patent Application Publication
(10) Pub. No.: US 2011/0229776 A1
(43) Pub. Date: Sep. 22, 2011

(54) METHOD FOR IMMOBILIZING ENZYME ON ELECTRODE FOR FUEL CELL, FUEL CELL, METHOD FOR MANUFACTURING FUEL CELL, ELECTRODE FOR FUEL CELL, AND METHOD FOR MANUFACTURING ELECTRODE FOR FUEL CELL

(75) Inventors: Hideyuki Kumita, Kanagawa (JP); Takaaki Nakagawa, Kanagawa (JP); Hideki Sakai, Kanagawa (JP); Masaya Kakuta, Tokyo (JP); Yuichi Tokita, Kanagawa (JP)

(73) Assignee: SONY CORPORATION, Tokyo (JP)

(21) Appl. No.: 13/130,475

(22) PCT Filed: Nov. 24, 2009
(86) PCT No.: PCT/JP2009/069803

§ 371 Date: May 20, 2011

(30) Foreign Application Priority Data
Nov. 25, 2008 (JP) 2008-299151

(51) Int. Cl. H01M 4/88; H01M 8/00; H01M 8/16; H01M 4/90; C12N 9/00; C12N 9/02

(52) U.S. Cl. 429/401; 429/535; 429/531; 435/183; 435/189

(57) ABSTRACT

Provided are an enzyme immobilizing method, a fuel cell and an electrode for the fuel cell which employ the enzyme immobilizing method, and a method for manufacturing the fuel cell and the electrode. The enzyme immobilizing method prevents reduction in enzyme activity when the enzyme is immobilized on the electrode, so as to make it possible to obtain a high catalyst current value. In the method for immobilizing an enzyme on the electrode used in the fuel cell, an enzyme variant with at least one amino acid residue being deleted, substituted, added, or inserted in a wild-type amino acid sequences is used as the enzyme, and the enzyme variant increases in activity through heat treatment. The immobilization is performed within a temperature range which makes it possible to increase the activity of the enzyme variant.

### CROSS REFERENCE TO RELATED APPLICATIONS

The present application is a National Stage of International Application No. PCT/JP2009/069803 filed on Nov. 24, 2009, which claims priority to Japanese Patent Application No. 2008-299151 filed on Nov. 25, 2008, the entire contents of which are being incorporated herein by reference.

### SEQUENCE LISTING

This disclosure includes a sequence listing submitted as a text file pursuant to 37 C.F.R. §1.52(e)(v) named CI-#9209784-v1-Sequence_Listing_ST25.TXT, created on May 19, 2011, with a size of 33,147 bytes, which is incorporated herein by reference. The attached sequence descriptions and Sequence Listing comply with the rules governing nucleotide and/or amino acid sequence disclosures in patent applications as set forth in 37 C.F.R. §§1.821-1.825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984). The symbols and format used for nucleotide and amino acid sequence data comply with the rules set forth in 37 C.F.R. §1.822.

### BACKGROUND

A fuel cell has a structure in which a cathode (oxidant electrode) and an anode (fuel electrode) are opposed to each other with an electrolyte (protonic conductor) in between. In fuel cells of related art, a fuel (hydrogen) supplied to the anode is oxidized and separated into electrons and protons (H⁺). The electrons are passed to the anode and the H⁺ moves through the electrolyte to the cathode. In the cathode, the H⁺ reacts with oxygen supplied from the outside and the electrons sent from the anode via an external circuit to produce water (H₂O).

A fuel cell is a high-efficiency electricity generating device that directly converts the chemical energy of a fuel to electric energy, and by which the chemical energy of fossil fuel such as natural gas, oil, and coal is obtained as electric energy at high conversion efficiency regardless of the place and time the device is used. A fuel cell is therefore being widely applied to large-scale power generation, a power source for driving a vehicle, a portable power source of a personal computer, a mobile device, or the like, and the like.

However, in the case of using, particularly, gas as the fuel, the gas has to be handled with care. In particular, in the case of using the fuel by heating it to high temperature, a safety issue arises. There is also an issue from the viewpoint of manufacturing cost for a reason that a catalyst of an expensive noble metal such as platinum (Pt) is necessary.

To address the issues, in recent years, paying attention to the fact that the biological metabolism of a living organism relates to a high-efficient energy conversion mechanism, a fuel cell (also called “biofuel cell” or “enzyme cell”) in which the fuel is separated to H+ and electrons by using enzyme is being developed (see patent documents 1 and 2).

In an enzyme cell, alcohols such as methanol and ethanol or sugars such as glucose may be used as the fuel. Consequently, the safety issue as described above is solved. In addition, by using excellent catalyst activity of enzyme, a fuel cell can be manufactured at low cost without using a noble metal catalyst.

To practically use the enzyme cell, a technique for stably obtaining a sufficient output has to be established. At present, technical developments for making the enzyme to be immobilized on the electrode act more stably against changes in the environment to maintain high activity are being actively promoted.

### CITATION LIST

### Patent Documents

Patent document 1: Japanese Unexamined Patent Application Publication No. 2004-71559

Patent document 2: Japanese Unexamined Patent Application Publication No. 2005-13210

To obtain a high output in an enzyme cell, by immobilizing the enzyme onto an electrode at high density, electrons generated by the enzyme reaction have to be efficiently taken in the electrode.

Usually, at the time of immobilizing the enzyme to the electrode, a carbon material or the like is immersed in an enzyme solution. After that, by evaporating moisture by drying, the enzyme is immobilized on the electrode material.

Generally, the enzyme has lowest stability in the solution state and is easily deactivated. Therefore, after the electrode material is immersed in the enzyme solution, deactivation of the enzyme has to be prevented by evaporating the moisture by prompt drying. On the other hand, the enzyme is easily denatured by heat, so that the drying has to be performed under a low temperature condition of room temperature or about 30° C. also in the case of using a drier.

In the techniques of related art, it is difficult to promptly dry the electrode immersed in the enzyme solution. Deterioration in the enzyme activity at the time of immobilization to the electrode is the bottleneck at the time of obtaining a high catalyst current value and a high output in an enzyme cell.

### SUMMARY

The present disclosure relates to a method for immobilizing enzyme on an electrode for a fuel cell. More particularly, the disclosure relates to an enzyme immobilizing method for immobilizing enzyme whose activity is increased by heat treatment on an electrode in a temperature range of increasing the activity. Further, the disclosure relates to a fuel cell and an electrode for a fuel cell using the enzyme immobilizing method and methods for manufacturing the fuel cell and the electrode for the fuel cell.

Therefore, it is a main object of the disclosure to provide a method of immobilizing enzyme to an electrode for a fuel cell, by which an electrode expressing a high catalyst current value is obtained without causing decrease in enzyme activity at the time of immobilizing the enzyme to the electrode.

It is another object of the disclosure to provide a fuel cell using the enzyme immobilizing method of the disclosure, a method of manufacturing the fuel cell, an electrode for a fuel cell, and a method of manufacturing the electrode.

To solve the above-mentioned problems, the disclosure provides an enzyme immobilizing method for immobilizing enzyme to an electrode used for a fuel cell, characterized in that as the enzyme, an enzyme variant obtained by deletion, substitution, addition, or insertion of at least one amino acid residue in a wild-type amino acid sequence and having a characteristic that its activity increases through heat treatment is immobilized in a temperature range in which the activity can be increased.

In the enzyme immobilizing method, as enzyme to be immobilized on a cathode, variant heat-resistant bilirubin oxidase, particularly, a variant heat-resistant bilirubin oxidase obtained by deletion, substitution, addition, or insertion of at least one amino acid residue in a wild-type amino acid sequence of bilirubin oxidase derived from imperfect filamentous fungus, Myrothecium verrucari, represented by sequence number 1 can be used.

Further, in this case, preferably, a variant bilirubin oxidase represented by sequence number 2 in which phenylalanine at the 225th position from an N-terminal of the wild-type amino acid sequence is substituted with valine (F225V), asparagine acid at the 322nd position is substituted with asparagine (D322N), and methionine at the 468th position is substituted with valine (M468V); a variant bilirubin oxidase represented by sequence number 3 in which phenylalanine at the 225th position from an N-terminal of the wild-type amino acid sequence is substituted with valine (F225V), asparagine acid at the 370th position is substituted with tyrosine (D370Y), and leucine at the 476th position is substituted with proline (L476P); a variant bilirubin oxidase represented by sequence number 4 in which alanine at the 264th position from an N-terminal of the wild-type amino acid sequence is substituted with valine (A264V), alanine at the 418th position is substituted with threonine (A418T), and leucine at the 476th position is substituted with proline (L476P); a variant bilirubin oxidase represented by sequence number 5 in which alanine at the 264th position from an N-terminal of the wild-type amino acid sequence is substituted with valine (A264V), arginine at the 437th position is substituted with histidine (R437H), and leucine at the 476th position is substituted with proline (L476P); and/or a variant bilirubin oxidase represented by sequence number 6 in which alanine at the 103rd position from an N-terminal of the wild-type amino acid sequence is substituted with proline (A103P), alanine at the 264th position from the N-terminal is substituted with valine (A264V), tyrosine at the 270th position is substituted with asparagine acid (Y270D), and leucine at the 476th position is substituted with proline (L476P) is used, and immobilization is performed in a temperature range from 20° C. to 65° C., both inclusive.

Moreover, the disclosure provides a method of manufacturing a fuel cell having a structure in which electrodes are opposed to each other with a proton conductor therebetween, characterized in that the step of immobilizing, to the electrode, an enzyme variant obtained by deletion, substitution, addition, or insertion of at least one amino acid residue in a wild-type amino acid sequence and having a characteristic that its activity increases through heat treatment in a temperature range in which the activity can be increased is included, and a fuel cell obtained by the manufacturing method.

Further, the disclosure provides a method of manufacturing an electrode for use in a fuel cell, characterized in that the step of immobilizing, to the electrode, an enzyme variant obtained by deletion, substitution, addition, or insertion of at least one amino acid residue in a wild-type amino acid sequence and having a characteristic that its activity increases through heat treatment in a temperature range in which the activity can be increased is included, and an electrode for a fuel cell obtained by the manufacturing method.

In the disclosure, “bilirubin oxidase” denotes enzyme for catalyzing reaction of oxidizing bilirubin to biliverdin, which is a kind of enzymes belonging to multicopper oxidase (generic name of enzymes having a plurality of copper ions in the active center).

The present disclosure provides an enzyme immobilizing method capable of obtaining an electrode expressing a high catalyst current value without causing decrease in enzyme activity at the time of immobilizing enzyme to the electrode and achieving large shortening of time of the electrode manufacturing process and higher efficiency. In addition, by the enzyme immobilizing method, even when a low-purity or unrefined enzyme is used for immobilization, the objects other than the target enzyme can be deactivated by the heat treatment at the time of immobilization. Consequently, hindrance of activation of the enzyme by impurity and deterioration in stability is prevented, and an electrode expressing a high catalyst current value can be obtained.

Additional features and advantages are described herein, and will be apparent from the following Detailed Description and the figures.

### BRIEF DESCRIPTION OF THE FIGURES

FIG. 1 is a diagram illustrating an example of thermal stabilization screening and showing a state of color generation of ABTS (one hour after reaction start).

FIG. 2 is a diagram illustrating a UV-vis spectrum of the variant recombinant BO.

FIG. 3 is a diagram illustrating an example (electrode 1) of voltermmogram measured in Example 5.

### DETAILED DESCRIPTION

In an enzyme immobilizing method according to the disclosure, any of the following enzymes having the characteristic that activity increases through heat treatment is used.

As an enzyme immobilized on a cathode, an oxidase which is, for example, multicopper oxidase such as bilirubin oxidase, laccase, or ascorbic acid oxidase is used.

The enzyme immobilized on an anode is selected according to a fuel used. For example, in the case of using a monosaccharide such as glucose as the fuel, oxidase that promotes oxidation of the monosaccharide and decomposes it is used. Usually, in addition to this, a coenzyme oxidase which changes a coenzyme reduced by the oxidase back to an oxidant is used. By the action of the coenzyme oxidase, electrons are generated when the coenzyme changes back to the oxidant and are transferred from the coenzyme oxidant to the electrode via an electron mediator.

As the oxidase, for example, an NAD-dependent glucose dehydrogenase such as glucose dehydrogenase (GDH) is used. As the coenzyme oxidant, for example, an NADH oxide reductase such as diaphorase is used.

Further, in the case of using a polysaccharide as the fuel, in addition to the oxidase and the coenzyme oxidase, a degrading enzyme which promotes degradation such as hydrolytic degradation to generate a monosaccharide such as glucose is also immobilized. Note that it is assumed here that a polysaccharide denotes a polysaccharide in a broad sense, refers to all of carbohydrates that generate a monosaccharide of two or more molecules by hydrolytic degradation and includes an oligosaccharide such as disaccharide, trisaccharide, or tetrasaccharide.

As the degrading enzyme, for example, amylase, glucosidase, dextrinase, sucrase, lactase, or cellulase is used.

In the enzymes, particularly, an enzyme having the characteristic that the activity increases through heat treatment is obtained by making a base sequence of a gene that codes each of enzyme proteins artificially mutated and expressed in a cell of escherichia coli, an enzyme variant obtained by deletion, substitution, addition, or insertion of at least one amino acid residue in a wild-type amino acid sequence is artificially prepared and obtained by performing screening. The base sequence of a gene is altered by a method capable of introducing a gene variant at random such as error prone PCR. An enzyme variant is obtained by introducing the altered gene to a host cell by a known method, extracting protein from the cell, and performing purification by affinity column chromatography. Then, by performing screening on the obtained enzyme variant obtained by a thermo stabilization test (refer to examples), the enzyme variant having the above-described characteristics can be obtained.

In the enzyme immobilizing method according to the disclosure, the enzyme variant obtained by the screening is immobilized on an electrode in a temperature range in which the enzyme activity is increased. The upper limit value of the temperature range is properly set for each of enzyme variants on the basis of a result of the thermo stabilizing test. The lower limit value of the temperature range is 20° C. or higher as normal environment temperature (room temperature), preferably, 30° C., and more preferably, 40° C.

An enzyme variant can be immobilized on an electrode according to an existing method by preparing an enzyme variant solution, immersing the electrode in the solution, and drying the electrode by using a drier or the like. In the enzyme immobilizing method according to the disclosure, the dry immobilization can be performed in the above temperature range. The lower limit value of the temperature range is a value as described above. The hither the temperature at which the dry immobilization is performed is, the quicker moisture in the enzyme solution adhered to the electrode is evaporated. Time of the drying is properly set to sufficiently evaporate the moisture in the solution within heat treatment time which is confirmed as time in which the enzyme activity of a variant can be increased by the thermo stabilization test. The temperature and time of the heat treatment are, for example, in the examples 60° C. and one hour or less, or 65° C. and 30 minutes or less.

The enzyme variant may be immobilized by dropping, spraying, or applying a solution to an electrode and, similarly, by drying. Further, at the time of immobilization, various immobilization agents may be used. Preferably, a polyion complex formed by using polycation such as poly-L-lycine (PLL) and its salt or polyanion such as polyacrylic acid (for example, sodium polyacrylate (PAAcNa)) and its salt is used.

As described above, by the enzyme immobilizing method according to the disclosure, moisture in the enzyme solution is promptly evaporated. Consequently, time during which the enzyme is in an unstable solution state is shortened, and deactivation of the enzyme can be prevented. In addition, different from enzymes usually used, the enzyme activity of the enzyme variant used in the disclosure increases through heat treatment, so that the enzyme after drying immobilization expresses enzyme activity higher than before immobilization. Therefore, in the electrode after enzyme is immobilized, a high catalyst current value can be obtained.

Concretely, the inventors of the present disclosure have obtained the following variants as variant bilirubin oxidase whose activity increases through heat treatment, obtained by performing screening in a thermo stabilization test on variant bilirubin oxidase formed by introducing random mutation in a wild-type amino acid sequence (refer to sequence number 1) of bilirubin oxidase derived from imperfect filamentous fungus, Myrothecium verrucaria (hereinbelow, called M. verrucaria), and causing expression and purification.

A variant bilirubin oxidase represented by sequence number 2 in which phenylalanine at the 225th position from an N-terminal of the wild-type amino acid sequence is substituted with valine (F225V), asparagine acid at the 322nd position is substituted with asparagine (D322N), and methionine at the 468th position is substituted with valine (M468V).

A variant bilirubin oxidase represented by sequence number 3 in which phenylalanine at the 225th position from an N-terminal of the wild-type amino acid sequence is substituted with valine (F225V), asparagine acid at the 370th position is substituted with tyrosine (D370Y), and leucine at the 476th position is substituted with proline (L476P).

A variant bilirubin oxidase represented by sequence number 4 in which alanine at the 264th position from an N-terminal of the wild-type amino acid sequence is substituted with valine (A264V), alanine at the 418th position is substituted with threonine (A418T), and leucine at the 476th position is substituted with proline (L476P).

A variant bilirubin oxidase represented by sequence number 5 in which alanine at the 264th position from an N-terminal of the wild-type amino acid sequence is substituted with valine (A264V), arginine at the 437th position is substituted with histidine (R437H), and leucine at the 476th position is substituted with proline (L476P).

A variant bilirubin oxidase represented by sequence number 6 in which alanine at the 103rd position from an N-terminal of the wild-type amino acid sequence is substituted with proline (A103P), alanine at the 264th position from the N-terminal is substituted with valine (A264V), tyrosine at the 270th position is substituted with asparagine acid (Y270D), and leucine at the 476th position is substituted with proline (L476P).

In those variant bilirubin oxidases, the enzyme activity is held highly even by the heat treatment at 60° C. or 65° C., excellent heat resistance is expressed, and enzyme activity significantly higher than that of the wild-type enzyme is expressed by the heat treatment at the same temperature.

Therefore, at the time of immobilizing any of the enzyme variants to an electrode, by performing immobilization at 65° C. or less, more preferably, in a temperature range of 60° C. or less, a catalyst current value higher than that in the wild-type enzyme is obtained. In addition, the immobilization may be performed under temperature condition higher than 60° C. or 65° C. as long as the activity of the enzyme variant is increased.

By the above enzyme immobilizing method, as the electrode material for manufacturing the electrode and a fuel cell, a known material such as a carbon-based material may be used. Alternately, a porous conductive material containing skeleton made of a porous material and a material whose main component is a carbon-based material covering at least a part of the surface of the skeleton may be used.

The porous conductive material is obtained by coating at least a part of the surface of the skeleton made of the porous material with the material whose main component is a carbon-based material. Any porous material constructing the skeleton of the porous conductive material may be basically used as long as the skeleton is stably maintained even if the porosity rate is high and regardless of conductivity. As the porous material, suitably, a material having high porosity rate and high conductivity is used. As the porous material having high porosity rate and high conductivity, concretely, a metal material (metal or alloy), a carbon-based material whose skeleton is strengthened, or the like can be used. In the case of using a metal material as the porous material, there are various options since the state stability of the metal material varies according to the balance with use environments such as pH of a solution, potential, and the like. For example, a foam metal or foam alloy such as nickel, copper, silver, gold, nickel chrome alloy, or stainless steel is one of materials which are easily obtainable. As the porous material, other than the above-described metal materials and the carbon-based material, a resin material (for example, a sponge type) may be used. The porosity rate and the pore diameter (the minimum pore diameter) of the porous material are determined according to the porosity rate and the pore diameter necessary for the porous conductive material in balance with the thickness of the material whose main component is the carbon-based material, which is to be coated on the surface of the skeleton made of the porous material. The pore diameter of the porous material is generally 10 nm to 1 mm, both inclusive, typically, 10 nm to 600 μm, both inclusive.

On the other hand, as the material covering the surface of the skeleton, a material having conductivity and stability in operation potential assumed has to be used. As such a material, a material whose main component is a carbon-based material is used here. The carbon-based material has generally a wide potential window and, moreover, is chemically stable. The material whose main component is a carbon-based material is concretely a carbon-based material itself or a material whose main component is a carbon-based material and containing a small amount of a sub-material selected according to a characteristic necessary for the porous conductive material or the like. Concrete examples of the latter material include a material whose electric conductivity is improved by adding a high-conductive material such as metal to the carbon-based material, and a material to which a function other than conductivity is applied such as surface water repellency by adding a polytetrafluoroethylene-based material or the like to the carbon-based material. There are various kinds of the carbon-based materials. Any carbon-based material may be used. Not only simple carbon but also a material obtained by adding another element to carbon may be used. As the carbon-based material, particularly, a fine powder carbon material having high conductivity and large surface area is preferable. As the carbon-based material, concretely, for example, a material to which high conductivity is applied such as KB (Ketjen black), a functional carbon material such as carbon nanotube or fullerene, or the like may be used.

As a method of coating the material whose main component is the carbon-based material, any coating method may be used as long as it coats the surface of the skeleton made of the porous material by using a proper bonding agent as necessary. The pore diameter of the porous conductive material is selected to a degree that a solution including a substrate easily pass through the pore and is, generally, 9 nm to 1 mm both inclusive, more generally, 1 μm to 1 mm both inclusive, and further more generally, 1 to 600 μm both inclusive. In a state where at least a part of the surface of the skeleton made of the porous material is covered with the material whose main component is the carbon-based material, or in a state where at least a part of the surface of the skeleton made of the porous material is coated with a material whose main component is the carbon-based material, preferably, all of pores are conducted to one another, or clogging caused by a material whose main component is the carbon-based material is prevented.

An electronic mediator immobilized on the electrode may use one or more of 2-methyl-1,4-naphthoquinone (VK3), 2-amino-3-carboxy-1,4-naphthoquinone (ACNQ), 2-amino-1,4-naphthoquinone (ANQ), and the like for the anode and may use one or more of potassium ferricyanide, and the like for the cathode as necessary.

In the case of using a polysaccharide, as the fuel, for example, starch, amylose, amylopectin, glycogen, cellulose, maltose, sucrose, lactose or the like may be used. Any one of these polysaccharides is composed of two or more monosaccharides and contains glucose as a monosaccharide of a bond unit. In addition, amylose and amylopectin are components in starch. Starch is a mixture of amylose and amylopectin. When glucoamylase is used as a degrading enzyme for a polysaccharide and glucose dehydrogenase is used as an oxidase for decomposing a monosaccharide, if a polysaccharide which can be decomposed to glucose by glucoamylase, for example, any one of starch, amylose, amylopectin, glycogen, and maltose, is contained, it may be used as the fuel. Note that glucoamylase is a degrading enzyme which hydrolyzes α-glucan such as starch to produce glucose, and glucose dehydrogenase is an oxidase which oxidizes β-D-glucose to D-glucono-δ-lactone.

For a proton conductor, as an electrolyte containing a buffer material, for example, an electrolyte may be used, which contains dihydrogen phosphate ion (H₂PO₄⁻), 2-amino-2-hydroxymethyl-1,3-propanediol (abbreviated as “tris”), 2-(N-morpholino)ethanesulfonic acid (MES), cacodylic acid, carbonic acid (H₂CO₃), hydrogen citrate ion, N-(2-acetamido)iminodiacetic acid (ADA), piperazine-N,N′-bis(2-ethanesulfonic acid) (PIPES), N-(2-acetamido)-2-aminoethanesulfonic acid (ACES), 3-(N-morpholino)propanesulfonic acid (MOPS), N-2-hydroxyethylpiperazine-N′-2-ethanesulfonic acid (HEPES), N-2-hydroxyethylpiperazine-N′-3-propanesulfonic acid (HEPPS), N-[tris(hydroxymethyl)methyl]glycine (abbreviated as “tricine”), glycylglycine, or N,N-bis(2-hydroxyethyl)glycine (abbreviated as “bicine”). Generally, any buffer material having pKₐ of 6 to 9 both inclusive may be used. It is effective to set the concentration of the buffer material included in the electrolyte to 0.2M to 2.5M both inclusive, preferably, 0.2M to 2M both inclusive, more preferably, 0.4M to 2M both inclusive, and further more preferably, 0.8M to 1.2M both inclusive so that sufficient buffer capacity is obtained in high output operation and the capability of the enzyme is fully displayed. The pH of the electrolyte is preferably around seven and, generally, may be 1 to 14 both inclusive.

In the method for manufacturing electrode and the method for manufacturing fuel cell according to the disclosure, by arbitrarily selecting any of the concrete examples in the configurations by using the above enzyme immobilizing method, an electrode and a fuel cell can be obtained. In the electrode and the fuel cell, a high catalyst current value and high output based on the high enzyme activity of the immobilized enzyme can be obtained.

### EXAMPLES

### Example 1

### cDNA Cloning of Bilirubin Oxidase (BO) Derived from M. Verrucaria

1-1. Culture of M. Verrucaria and Isolation of Messenger RNA

A character of M. verrucaria NBRC (IFO) 6113 used in the present Example was purchased from Department of Biotechnology of National Institute of Technology and Evaluation. An obtained dry fungus body was suspended in a condensate (polypeptone: 0.5%, yeast extract: 0.3%, MgSO₄.7H₂O: 0.1%), and this suspension was inoculated on a potato dextrose agar (PDA) plate (potato dextrose: 2.4%, agarose: 1.5%). As a result of culture at room temperature for 5 to 7 days, the surface of the PDA plate was covered with white hypha. This was scraped by a spatula and preserved at −80° C. The yield of the fungus body was from 50 to 60 mg (wet weight) per PDA plate (having a diameter of 9 cm).

A messenger RNA (hereinafter, described as “mRNA”) was extracted as a total RNA (a mixture of mRNA, ribosomal RNA, and transfer RNA). 100 μg (quantitatively determined by UV absorption) of the total RNA was obtained from about 100 mg of lyophilized fungus powders of M. verrucaria, and the quarter amount of the total RNA was used as a template RNA of one reaction of the following reverse transcription PCR.

1-2. Preparation of BO Gene Fragment by Reverse Transcription PCR

The reverse transcription PCR was carried out by using an OneStep RT-PCR kit (manufactured by Qiagen Corporation) with the above total RNA as a template. A PCR primer for use in the reverse transcription PCR was designed as shown in the following Table 1 on the basis of a base sequence of circular DNA (cDNA) of BO previously stated.

TABLE 1
N-Terminal side, HindIII site (AAGCTT) inserted
5′-GGGAAGCTTATGTTCAAACACACACTTGGAGCTG-3′
(sequence number 8)
C-Terminal side, XbaI (TCTAGA) site inserted
5′-GGGTCTAGACTCGTCAGCTGCGGCGTAAGGTCTG-3′
(sequence number 9)

As a result of agarose gel electrophoresis of the obtained PCR product, a strong band was recognized around 1,700 bp. In view of the size of 1,700 bp, the fragment was estimated to be an amplified fragment containing the desired BO gene, and therefore the fragment was cut out from an agarose gel slab and used in the following process.

1-3. Integration of BO Gene Fragment into pYES2/CT Vector

The obtained amplified fragment of 1,700 bp was digested by restriction enzymes HindIII and XbaI and then coupled with a pYES2/CT plasmid vector (manufactured by Invitrogen Corporation) digested by the enzymes. On that occasion, an alkaline phosphatase derived from Calf intestine (manufactured by Takara Bio Inc.) was used for a process of dephosphorylating a 5′-protruding end of the pYES2/CT vector by the restriction enzyme treatment, and T4 DNA ligase (manufactured by Takara Bio Inc.) was used for a coupling reaction between the inserted fragment and the pYES2/CT vector.

A character of E. coli TOP10 (manufactured by Invitrogen Corporation) was transformed by a reaction product obtained, and inoculated on an LB/Amp agar plate (having a composition as shown in Table 2). After culture of one night, a colony of a transformant having drug resistance to ampicillin was obtained. The colony was cultured overnight on an LB/Amp medium of 3 ml, and a plasmid vector was isolated from the obtained fungus body.

TABLE 2

| | |
|---|---|
| Tryptophan | 1% |
| Yeast extract | 0.5% |
| Sodium chloride | 1% |
| ampicillin | 0.005% |

The base sequence of the inserted portion containing the BO gene of the obtained plasmid vector was found to have sequence number 7.

The base sequence represented in the sequence number 7 is 1,719 bp and corresponds to 572 amino acid residues. On the other hand, a BO derived from M. verrucaria of a maturation type is made of 534 amino acid residues (sequence number 1). Here, the 38 amino acid residues corresponding to the difference exists on the N-terminal side and are a signal peptide for governing the secretory of a protein existing on the C-terminal side. After translation, the portion is cleaved at the time of secretory.

1-4. Insertion of AAA Sequence

Next, a part of the base sequence of the plasmid vector prepared in 1-3 was modified so as to increase the expression amount of the recombinant protein. Concretely, three bases on the upstream side (5′-side) relative to a start codon (ATG) were changed as shown in Table 3. The change of these three bases was carried out by a Quick-Change Mutagenesis Kit (manufactured by Stratagene Corporation) using a PCR primer shown in Table 4. The detailed experimental procedures followed a manual attached to the product.

TABLE 3
Before modification:
5′- . . . ATTAAG AAATG TTCAAAC . . . 3′
(sequence number 10)
After modification:
5′- . . . ATTAAG AAAATG TTCAAAC . . . 3′
(sequence number 11)

TABLE 4
N-Terminal side:
5′-CTATAGGGAATATTAAGAAA ATG TTCAAACACACACTTG-3′
(sequence number 12)
C-Terminal side:
5′-CAAGTGTGTGTTTGAA CAT TTTCTTAATATTCCCTATAGTG-3′
(sequence number 13)

The verification of the base sequence was carried out in the entire region of the BO gene including the changed sites. It was verified that the base sequence was changed as designed. The plasmid vector after changing the sequence is hereinafter called “pYES2/CT-BO vector”.

### Example 2

### Construction of Secretory Expression System of Recombinant BO by S. Cerevisiae

2-1. Transformation of S. Cerevisiae by pYES2/CT-BO Vector

Next, S. cerevisiae was transformed by using the above-described pYES2/CT-BO vector. As S. cerevisiae, a character of INVScl (manufactured by Invitrogen Corporation) which is marketed along with the pYES2/CT vector was used. In this case, S. cerevisiae was transformed by the lithium acetate method. With respect to the detailed experimental procedures, a manual attached to the pYES2/CT vector was used for reference. To select transformed yeast, an SCGlu agar plate (having a composition shown in Table 5) was used.

TABLE 5

| | |
|---|---|
| Yeast Nitrogen Base (YNB) | 0.17% |
| (NH₄)₂SO₄ | 0.5% |
| L-Arginine | 0.01% |
| L-Cysteine | 0.01% |
| L-Leucine | 0.01% |
| L-Lysine | 0.01% |
| L-threonine | 0.01% |
| L-Tryptophan | 0.01% |
| L-Asparagine acid | 0.005% |
| L-Histidine | 0.005% |
| L-Isoleucine | 0.005% |
| L-Methionine | 0.005% |
| L-Phenylalanine | 0.005% |
| L-Proline | 0.005% |
| L-Serine | 0.005% |
| L-Tyrosine | 0.005% |
| L-Valine | 0.005% |
| Adenine | 0.01% |
| D-Glucose | 2% |
| Agarose | 2% |

Secretory Expression of Recombinant BO

The colony of the transformant of S. cerevisiae by the pYES2/CT-BO vector was inoculated on 15 ml of an SCGlu liquid culture medium and cultured with shaking at 30° C. for 14 to 20 hours. The resulting fungus body was once precipitated by centrifugation (1,500×g at room temperature for 10 minutes).

Here, after discarding the SCGlu liquid culture medium, the obtained fungus body was added in 50 ml of an SCGal medium (having a composition shown in Table 6) so that a turbidity (OD₆₀₀) becomes about 0.5. The resultant was cultured with shaking at 25° C. for 10 to 14 hours. After the culture, the fungus body was removed by centrifugation, the residual culture solution was concentrated to a degree of about 5 ml and dialyzed against a 20 mM sodium phosphate buffer (pH: 7.4).

TABLE 6

| | |
|---|---|
| Yeast nitrogen base (YNB) | 0.17% |
| (NH₄)₂SO₄ | 0.5% |
| L-Arginine | 0.01% |
| L-Cysteine | 0.01% |
| L-Leucine | 0.01% |
| L-Lysine | 0.01% |
| L-Threonine | 0.01% |
| L-Tryptophan | 0.01% |
| L-Asparagine acid | 0.005% |
| L-Histidine | 0.005% |
| L-Isoleucine | 0.005% |
| L-Methionine | 0.005% |
| L-Phenylalanine | 0.005% |
| L-Proline | 0.005% |
| L-Serine | 0.005% |
| L-Tyrosine | 0.005% |
| L-Valine | 0.005% |
| Adenine | 0.01% |
| D-Galactose | 2% |
| D-Raffinose | 1% |
| Glycine | 1% |
| CuSO₄•5H₂O | 0.003% |

The recombinant BO was purified by Ni-NTA affinity chromatography (His-trap HP (1 ml), manufactured by Amersham Biosciences K.K.). The purification was performed according to the purification method in a manual attached to the product. The recombinant BO obtained after the purification was verified to have a purity of 100 by SDS-PAGE or the like. The yield of the recombinant BO was respectively 0.36 mg in 1 L culture.

### Example 3

### Thermal Stabilization Screening of Recombinant BO by Evolutionary Molecular Engineering Method

Subsequently, the recombinant BO was subjected to thermal stabilization screening by the evolutionary molecular engineering method. Concretely, insertion of random mutation using Error-prone PCR, preparation of a BO gene library of a variant, and transformation of S. cerevisiae by the variant gene library were carried out and, after that, the thermal stabilization screening using a 96-well plate was performed.

3-1. Insertion of Random Mutation Using Error-Prone PCR

The insertion of random mutation by Error-prone PCR was carried out using a pYES2/CT-BO vector as a template. The PCR primer on the N-terminal side used in the example was designed so as to contain the only BgIII site (AGATCT) existing on the downstream side of the 218 base pairs relative to the start codon. Also, the C-terminal side was designed in the following manner so as to include the XbaI site (TCTAGA) (see Table 7).

TABLE 7
N-Terminal side, BglII (AGATCT) site inserted
5′-GTAACCAATCCTGTGAATGGACAAG AGATCT GG-3′
Sequence number 14
C-Terminal side, XbaI (TCTAGA) site inserted
5′-GGGATAGGCTTACCTTCGAAGGGCCC TCTAGA CTC-3′
Sequence number 15

The Error-prone PCR was carried out by a GeneMorph PCR Mutagenesis Kit (manufactured by Stratagene Corporation) by using the primers. Reaction conditions were set with reference to the manual attached to the kit.

An obtained PCR product was analyzed by agarose gel electrophoresis and a PCR fragment of about 1,500 bp was obtained. The frequency of mutation calculated from the yield of the obtained PCR product was 1.5 sites per 1,000 bp with reference to the calculation method in the manual attached to the kit.

3-2. Preparation of BO Gene Library of Variant

The BO gene fragment having mutation randomly inserted thereinto and prepared in 3-1 was integrated in the BgIII-XbaI site of the pYES2/CT-BO vector in a manner similar to the method described in 1-3 and a character of E. coli To P10 was transformed. Here, a plasmid library including about 6,600 transformed colonies, namely about 6,600 kinds of variant genes was obtained.

3-3. Transformation of S. Cerevisiae by Variant BO Gene Library

By the method described in 3-2, a character of S. cerevisiae INV Scl (manufactured by Invitrogen Corporation) was transformed by the variant gene library. A competent cell of S. cerevisiae INVScl was prepared by the lithium acetate method. The obtained transformed library was subjected to thermal stabilization screening using a 96-well plate.

3-4. Thermal Stabilization Screening Experiment Using 96-Well Plate

An SCGlu medium was dispensed by 150 ml to a 96-well plate. After that, one colony of the prepared transformed yeast library was inoculated in each well. The medium was cultured with shaking at 27° C. for 20 to 23 hours. After the culture, the turbidity of the wells became substantially constant.

At this stage, each 96-well plate was once subjected to centrifugation (1,500×g, 20° C., 10 minutes), thereby once precipitating the fungus body. The SCGlu medium was completely removed in such a manner that the fungus body precipitated on the bottom of each well was not disturbed. An amount of 180 ml of an SCGal medium was dispensed to the plate and was further cultured with shaking at 27° C. for eight hours. After the culture, the centrifugation (1,500×g, 20° C., 10 minutes) was again carried out to precipitate the fungus bodies. 100 ml of supernatant solution was transferred to a new 96-well plate. In the case of performing heat treatment, a sample solution on this 96-well plate was sealed with a cellophane tape and then left in a dry oven of 80° C. for 15 minutes. After the heat treatment, the sample solution was rapidly cooled on an ice bath for five minutes and then left at room temperature for 15 minutes. An equal amount of a 20 mM ABTS solution (100 mM Tris-HCl, pH: 8.0) was mixed therewith. The state that the solution in the wells exhibits green color in association with the reaction with ABTS was observed for one hour since start of the reaction. The solutions exhibiting color more than the wild type in reaction with ABTS were picked up, and corresponding fungus bodies were stored as 20 glycerol stocks at −80° C.

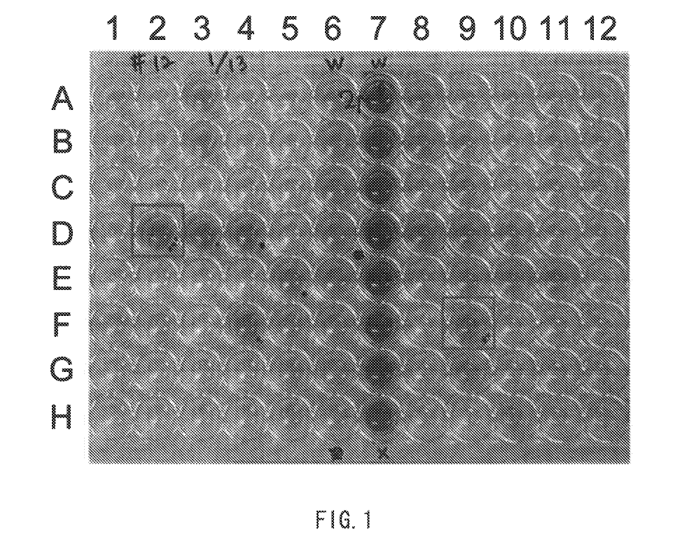

FIG. 1 shows one example of thermal stabilization screening. FIG. 1 shows the state of color representation of ABTS one hour after the start of the reaction. All of the wells in the central two columns (sixth and seventh columns from the left side) are of the wild-type recombinant BO as a comparison. The wild-type recombinant BO in the wells of the sixth column was subjected to the heat treatment like those in the other wells. The seventh column relates to a comparison in which the wild-type recombinant BO is not subjected to the heat treatment.

It is understood from FIG. 1 that the ABTS in the wells each surrounded by a square gives color stronger than that in any of the wild type in the sixth column. It is considered that in these wells, variant BO whose thermal stability is improved as compared with the wild-type recombinant BO is expressed.

In the present example, the thermal stabilization screening as described in 3-4 was performed on 4,000 samples in 50 96-well plates in total, and 26 transformed yeasts which are considered to have expressed the heat-resistant BO variant were chosen.

Plasmid vectors were extracted with the obtained 26 transformed yeasts, and the base sequence of the BO gene region was analyzed. As a result, it was found that the following 26 kinds of mutations were inserted in the BO gene. That is, the following mutations were recognized: a mutation (Q49K) in which glutamine at the 49th position from the N-terminal of the wild-type amino acid sequence is substituted with lysine; similarly, a mutation (Q72E) in which glutamine at the 72nd position is substituted with glutamic acid; a mutation (V81L) in which valine at the 81st position is substituted with leucine; a mutation (A103P) in which alanine at the 103rd is substituted with proline; a mutation (Y121S) in which tyrosine at the 121st position is substituted with serine; a mutation (R147P) in which arginine at the 147th position is substituted with proline; a mutation (A185S) in which alanine at the 185th position is substituted with serine; a mutation in which proline at the 210th position is substituted with leucine (P210L); a mutation (F225V) in which phenylalanine at the 225th position is substituted with valine; a mutation (G258V) in which glycine at the 258^{th} position is substituted with valine; a mutation (A264V) in which alanine at the 264th position is substituted with valine; a mutation (Y270D) in which tyrosine at the 270th position is substituted with asparagine acid; a mutation (S299N) in which serine at the 299th position is substituted with asparagines; a mutation (D322N) in which asparagine acid at the 322nd position is substituted with asparagines; a mutation (N335S) in which asparagine at the 335th position is substituted with serine; a mutation (R356L) in which arginine at the 356th position is substituted with leucine; a mutation (P359S) in which proline at the 359th position is substituted with serine; a mutation (D370Y) in which asparagine acid at the 370th position is substituted with tyrosine; a mutation (V371A) in which valine at the 371st position is substituted with alanine; a mutation (V381L) in which valine at the 381st position is substituted with leucine; a mutation (A418T) in which alanine at the 418th position is substituted with threonine; a mutation (P423L) in which proline at the 423rd position is substituted with leucine; a mutation (R437H) in which arginine at the 437th position is substituted with histidine; a mutation (M468V) in which methionine at the 468th position is substituted with valine; a mutation (L476P) in which leucine at the 476th position is substituted with proline; and a mutation (V513L) in which valine at the 513rd position is substituted with leucine.

### Example 4

### Abundant Expression by Heat-Resistant Variant Pichia Methanolica

In order to achieve abundant expression of the 26 kinds of heat-resistant variant candidates discovered by the thermal stabilization screening, a secretory expression system of recombinant BO using a yeast Pichia methanolica (hereinafter referred to as “P. methanolica”) was newly constructed, thereby attempting to achieve abundant expression of the wild-type and heat-resistant variant candidates.

4-1: Preparation of pMETaB-BO Vector and Transformation of P. Methanolica by the Vector

First, an expression vector to be used in an expression system of P. methanolica was prepared. Since a secretory signal: α-factor derived from S. cerevisiae is contained in a pMETaB vector (manufactured by Invitrogen Corporation) used herein, a gene corresponding to a maturation BO was inserted on the downstream. The amplification of the maturation BO gene region by PCR was carried out by using the pYES2/CT-BO vector as a template and using primers as shown in the following Table 8.

TABLE 8
N-Terminal side, EcoRI (GAATTC) site inserted
5′-GGGAATTCTTGCCCAGATCAGCCCACAGTATC-3′
(sequence number 16)
C-Terminal side, Termination codon, SpeI (ACTAGT)
site inserted
5′-GGGACTAGTCACTCGTCAGCTGCGGCGTAAGG-3′
(sequence number 17)

The obtained amplified fragment of 1,500 bp was digested by restriction enzymes EcoRI and SpeI and then coupled with a pMETaB vector digested by the enzymes. On the occasion of the coupling reaction, the reaction product was subjected to the same treatment as that described in 1-3. With respect to the pMETaB vector including the BO gene region produced (hereinafter referred to as “pMETaB-BO vector”), the base sequence of the inserted BO gene portion was confirmed. In the case of the variant BO, mutations were inserted in the prepared pMETaB-BO vector by QuickChange Mutagenesis Kits (manufactured by Invitrogen Corporation). The subsequent operations were similarly carried out irrespective of the wild type and the variant.

In addition to the foregoing pMETaB-BO vectors of the wild type and 26 kinds of heat-resistant variant candidates, a pMETaB-BO vector of a multiple variant obtained by combining two, three or four of the 26 kinds of heat-resistant variant candidates was similarly prepared, and the base sequence was confirmed.

P. methanolica was transformed by all of the prepared pMETaB-BO vectors. A character of PMAD11 (manufactured by Invitrogen Corporation) was used as P. methanolica. The transformation was performed according to the method described in the manual attached to the pMETaB vector. The transformed yeast was selected by an MD agar plate (refer to Table 9 for a composition). Competencies of the reaction were all up to 10/1 μg DNA and were substantially coincident with the values described in the manual.

TABLE 9

| | |
|---|---|
| Yeast Nitrogen Base (YNB) | 1.34% |
| Biotin | 0.00004% |
| D-Glucose | 2% |
| Agarose | 1.5% |

4-2: Abundant Expression of Recombinant BO by P. Methanolica

A colony of the transformed yeast on the MD medium obtained five to seven days after the transformation was cultured for one night in 3 mL of a BMDY medium (refer to Table 10 for a composition). A part of the obtained culture solution was again developed to an MD agar plate. A white purified colony obtained two to three days after this was used for the abundant expression of the next item.

TABLE 10

| | |
|---|---|
| Yeast extract | 1% |
| Peptone | 2% |
| Potassium phosphate buffer solution (pH: | 100 mM |
| 6.0) | |
| Yeast nitrogen base (YNB) | 1.34% |
| Biotin | 0.00004% |
| D-glucose | 2% |

Next, the process moves to an operation of abundant expression of recombinant BO by P. methanolica. The purified colony of the transformed yeast was inoculated in 50 mL of a BMDY liquid medium and cultured with shaking at 30° C. for one night. The OD₆₀₀ at this time was 2 to 5. Here, the obtained fungus body was once precipitated by centrifugation (1,500×g, room temperature, ten minutes), the BMDY liquid medium was removed, and only the fungus body was then suspended in 50 to 100 ml of a BMMY liquid medium (refer to Table 11 for a composition). The suspension was cultured with shaking at 27° C. for 24 hours. After that, methanol was added so that final concentration becomes 0.5%, and the mixture was further cultured under the same conditions for 24 hours. After performing the operation up to 96 hours, the fungus body was removed by centrifugation, and the residual culture solution was concentrated to a degree of about 5 to 10 ml and dialyzed against a 50 mM Tris-HCl buffer (pH 7.6).

TABLE 11

| | |
|---|---|
| Yeast extract | 1% |
| Peptone | 2% |
| Potassium phosphate buffer solution (pH: | 100 mM |
| 6.0) | |
| Yeast nitrogen base (YNB) | 1.34% |
| Biotin | 0.00004% |
| Methanol | 0.5% |
| CuSO₄•5H₂O | 0.003% |

4-3. Purification of Recombinant BO

Subsequently, the recombinant BO was purified by anion-exchange chromatography. A crude solution containing the recombinant BO prepared in the preceding process was purified by using an anion-exchange column (HiTrap Q HP, bed volume: 5 ml, manufactured by GE Healthcare Bioscience Corp.). The purification conditions in the report (Biochemistry, 38, 3034-3042 (1999)) were referred to.

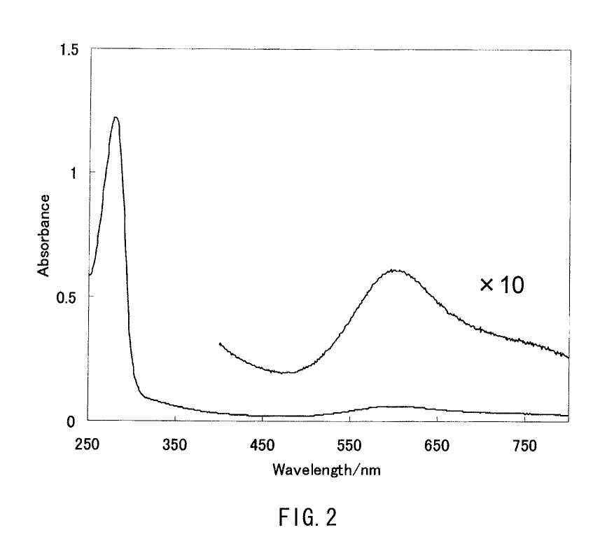

Next, the recombinant BO was purified by hydrophobic chromatography. A column used for the hydrophobic chromatography is a Toyopearl Butyl-650M column (100 ml, 20 mm×20 cm, manufactured by Tosoh Corporation). The purification condition in the report (Biochemistry, 44, 7004-7012 (2005)) was referred to. A UV-vis spectrum of the recombinant BO (A246V) obtained after the purification is illustrated in FIG. 2.

The spectral pattern of A264V illustrated in FIG. 2 perfectly coincides with that of a recombinant BO by P. pastris in the previous report (Protein Expression Purif., 41, 77-83 (2005)).

A final yield of the abundant culture by P. methanolica was 11.7 mg/1 L culture at maximum.

4-4: Evaluation of Heat Resistance

Next, the heat resistance of a recombinant BO by P. methanolica and a commercially available BO (manufactured by Amano Enzyme Inc.) was evaluated. The evaluation of the heat resistance was performed by comparison of the residual activity ratio after heat treatment. The “residual activity ratio after the heat treatment” here may be also called an “enzyme activity residual ratio” or “enzyme activity maintenance ratio” and is a value expressing a change in activity before and after heat treatment when a predetermined heat treatment is performed on enzyme. That is, it is a value obtained by measuring enzyme activity under the same conditions before and after the heat treatment and expressing the activity value in percentage after the heat treatment in comparison to the activity value before the treatment. The conditions of the “heat treatment” as criteria in the present disclosure are still standing process in a buffer solution at 60° C. for one hour or 65° C. for 30 minutes, and the ratio between the enzyme activity values before and after the heat treatment is expressed in percentage.

In BO activity measurement, ABTS was used as a substrate, a change in the absorbance at 730 nm with the progress of reaction (derived from an increase in the reaction product of ABTS) was followed. The measurement condition is shown in Table 12. In addition, during the activity measurement, the BO concentration was adjusted so that the change in the absorbance at 730 nm lies from about 0.01 to 0.2 per minute. The reaction was started by adding an enzyme solution (5 to 20 μL) in an ABTS-containing phosphate buffer solution (2,980 to 2,995 μL).

TABLE 12

| | |
|---|---|
| Buffer solution | 46.5 mM sodium phosphate aqueous |
| | solution (pH 7.0) |
| ABTS concentration | 2 mM (final concentration) |
| O₂ concentration | Saturated with air (210 · M, 25° C.) |
| Reaction temperature | 25° C. |

A heat resistance experiment was carried out on total 26 kinds of the heat-resistant BO variant candidates expressed by P. methanolica (Q49K, Q72E, V81L, A103P, Y121S, R147P, A185S, P210L, F225V, G258V, A264V, Y270D, S299N, D322N, N335S, R356L, P359S, D370Y, V371A, V381L, A418T, P423L, R437H, M468V, L476P, and V513L) and multiple variants each obtained by combining two, three or four of the candidates. For heat treatment on each of enzyme solutions, a method is employed in which 150 ml of an enzyme solution (100 mM potassium phosphate buffer (pH: 6.0)) dispensed in a 500-ml tube in an ice bath was rapidly moved onto a heat block which was set to 60° C. and left for one hour and, after that, the solution was rapidly returned in the ice bath. The results of the heat resistance verification experiment are summarized in Table 13.

The denaturation temperature Tₘ of 55 kinds of heat-resistant BO variant candidates subjected to evaluation of heat resistance was measured by differential scanning microcalorimetry (hereinafter referred to as “DSC” (Differential Scanning calorimetry)). VP-DSC manufactured by MicroCal, LLC was used as the DSC. An enzyme solution was used in an amount of 2.0 to 2.5 mg/ml, and temperature rise was carried out at a rate of 60° C. per hour. The results are summarized along with the results of the heat resistance verification experiment in Table 13.

TABLE 13

| Residual | | | |
|---|---|---|---|
| activity | | | |
| Denaturation | | | Triple variant |
| Temperature | Single variant | Double variant | quartet variant |
| 80% or higher | | Y121S/L476P, | Q49K/V371A/V513L, |
| 77° C. or | | A264V/R356L, | Y121S/D370Y/L476P, |
| higher | | A264V/L476P, | A185S/A264V/L476P, |
| | | D322N/M468V | K225V/D322N/M468V, |
| | | | A264V/R356L/L476P, |
| | | | A264V/S299N/L476P, |
| | | | A264V/V381L/L476P, |
| | | | A264V/A418T/L476P, |
| | | | A264V/R437H/L476P, |
| | | | A103P/A264V/Y270D/L476P |
| 50% or higher | Q72E, V81L, | Q49K/V371A, | Q72E/P210L/A264V, |
| 75° C. or | Y121S, | Q72E/P210L, | V81L/N335S/P423L, |
| higher | F225V, | Q72E/A264V, | F225V/D370Y/L476P |
| | A264V, | V81L/R147P, | |
| | D322N, | V81L/P423L, | |
| | R356L, | A185S/G258V, | |
| | P359S, | P210L/A264V, | |
| | D370Y, | F225V/D322N, | |
| | P423L, | F225V/L476P, | |
| | M468V, | N335S/P423L, | |
| | L476P, | R356L/L476P, | |
| | A103P, | V371A/V513L | |
| | S299N, | | |
| | V381L, | | |
| | A418T, | | |
| | R437H | | |
| 20% or higher | Q49K, | | |
| 72° C. or | R147P, | | |
| higher | A185S, | | |
| | P210L, | | |
| | G258V, | | |
| | N335S, | | |
| | V371A, | | |
| | V513I, | | |
| | V270D | | |
| Less than 20% | Wild type, | | |
| lower than 72° | commercial | | |
| C. | product | | |

In Table 13, some triple and quartet variants whose residue enzyme activity ratio is higher than 100% were observed. Consequently, next, the heat treatment temperature was raised to 65° C. and a similar heat-resistance verification experiment was made. The results of the experiment are summarized in Table 14. In Table 14, heat treatment time was 30 minutes.

TABLE 14

| Residual | | | Triple variant |
|---|---|---|---|
| activity | Single variant | Double variant | quartet variant |
| 100% or | | | F225V/D322X/M468V, |
| higher | | | F225V/D370Y/L476P, |
| | | | A264V/A418T/L476P, |
| | | | A264V/R437H/L476P, |
| | | | A103P/A264V/Y270D/L476P |
| 50% or higher | A103P, | Y121S/L476P, | Q49K/V371A/V513L, |
| | F225V, | P210L/A264V, | Q72E/P210L/A264V, |
| | A264V, | F225V/D322N, | V81L/N335S/P423L, |
| | S399N, | F225V/L476P, | Y121S/D370Y/L476P, |
| | D322N, | A264V/R356L, | A185S/A264V/L476P, |
| | D370Y, | A264V/L476P, | A264V/S299N/L476P, |
| | V381L, | D322N/M468V | A264V/R356L/L476P, |
| | A418T, | N335S/P423L, | A264V/V381L/L476P, |
| | P423L, | R356L/L476P | |
| | R437H, | | |
| | L476P | | |
| Less than 50% | Q49K, | Q49K/V371A, | |
| | Q72E, V81L, | Q72E/P210L, | |
| | Y121S, | Q72E/A264V, | |
| | R147P, | V81L/R147P, | |
| | A185S, | V81L/P423L, | |
| | P210L, | A185S/G258V, | |
| | G258V, | V371A/V513L | |
| | Y270D, | | |
| | N335S, | | |
| | R356L, | | |
| | P359S, | | |
| | Y371A, | | |
| | M468V, | | |
| | V5131 | | |
| | Wild types, | | |
| | Commercial | | |
| | products | | |

As summarized in Tables 13 and 14, remarkable drop in the residue enzyme activity ratio due to the heat treatment was recognized in the wild-type BO variants and commercially-available BO variants. On the other hand, it was confirmed that most of heat-resistant BO variant candidates highly maintain the enzyme activity even if the heat treatment was performed, and represent excellent heat resistance.

Further, as shown in Table 14, in the five heat-resistant BO variants F225V/D322N/M468V, F225V/D370Y/L476P, A264V/A418T/L476P, A264V/R437H/L476P, and A103P/A264V/Y270D/L476P, the residue enzyme activity ratio was increased by heat treatment at 65° C. for 30 minutes. It makes clear that the five variants have the outstanding characteristic that enzyme activity is increased by the heat treatment in addition to the heat resistance.

### Example 5

### Immobilization of Heat-Resistant BO Variant to Electrode and Performance Evaluation of Electrode

In the present example, one of the five heat-resistant BO variants whose enzyme activity was increased by the heat treatment (variant A103P/A264V/Y270D/L476P) was immobilized together with a mediator (K₃[Fe(CN)₆]) to manufacture an electrode. A catalyst current value of the manufactured electrode was measured by using a rotating electrode device to perform the performance evaluation of the electrode for a fuel cell. The heat-resisting BO variants were immobilized by the two methods: the method according to the present disclosure; and the existing method. The performances of the electrodes obtained by the methods were compared with each other. Further, on the commercial BO, similarly, immobilization was performed by the two methods and the performances of the electrodes obtained were compared with each other.

5-1. Manufacture of Electrodes

(1) Electrode 1 (Commercial BO, Existing Immobilizing Method)

Into two 1-cm-square sheets of carbon felt (CF2), 80 μl of a commercial BO solution (Amano Enzyme Inc.) (with a 46.5 mM sodium phosphate aqueous solution as a buffer solution), 80 μl of an immobilizer poly-L-lysine (PLL) 2 wt % solution, and 80 μl of a K₃[Fe(CN)₆] (hereinbelow, simply described as “FeCN”) were allowed to penetrate. As the commercial BO, a BO prepared by adding a freeze-dried product (having enzyme activity of 2.5 U/mg) in a 50 mg/ml solution was used (as for definition of activity of enzyme, refer to the attached sheet of the manufacturer).

The CF2 to which the solutions were allowed to penetrate was dried by a drier at 30° C. for two hours to evaporate the moisture. After the drying, the CF2 which [symbol] s cut in pieces each having a diameter of 6 mm was physically immobilized to the tip of a PFC electrode having a diameter of 6 mm (a carbon part has a diameter of 3 mm) with a nylon net and an O ring, thereby obtaining an electrode 1.

(2) Electrode 2 (Commercial BO, the Immobilizing Method of the Present Disclosure)

In a manner similar to the electrode 1, the CF2 to which the solutions were allowed to penetrate was dried by a drier at 30° C. for one hour and, further, dried at 60° C. for one hour to evaporate the moisture. After the drying, the CF2 was cut into pieces, and each of the pieces was immobilized to the tip of a PFC electrode, thereby obtaining an electrode 2.

(3) Electrode 3 (Heat-Resistive BO Variant, the Existing Immobilizing Method)

Into two 1-cm-square sheets of carbon felt (CF2), 80 μl of a variant BO solution, 80 μl of an immobilizer poly-L-lysine (PLL) 2 wt % solution, and 80 μl of an FeCN 200 mM solution were allowed to penetrate. A variant BO solution was prepared so that its absorbance at 600 nm becomes almost the same as that of a commercial BO solution.

The CF2 to which the solutions were allowed to penetrate was dried by a drier at 30° C. for two hours to evaporate the moisture. After the drying, the CF2 was cut in pieces, and each piece was immobilized to the tip of a PFC electrode, thereby obtaining an electrode 3.

(4) Electrode 4 (Heat-Resistive BO Variant, the Immobilizing Method According to the Disclosure)

The CF2 to which the solutions were allowed to penetrate in a manner similar to the case of the electrode 3 was dried by a drier at 30° C. for one hour and, further, dried at 60° C. for one hour to evaporate the moisture. After the drying, the CF2 was cut in pieces, and each piece was immobilized to the tip of a PFC electrode, thereby obtaining an electrode 4.

5-2: Measurement of Catalyst Current Value

The cyclic voltammetry (CV) was measured by using a rotation electrode device on the electrodes 1 to 4. The CV measurement was carried out by a potential-scan in a potential region from the initial potential 0.6V (vs. Ag|AgCl) to −0.35V (vs. Ag|AgCl) at a velocity of 10 mV/s. As the buffer solution, a 46.5 mM sodium phosphate buffer solution (2 ml) was used. By making pure oxygen bubbling in the buffer solution, an oxygen saturation solution was obtained. The rotational speed of the electrode was set to 1000 rpm.

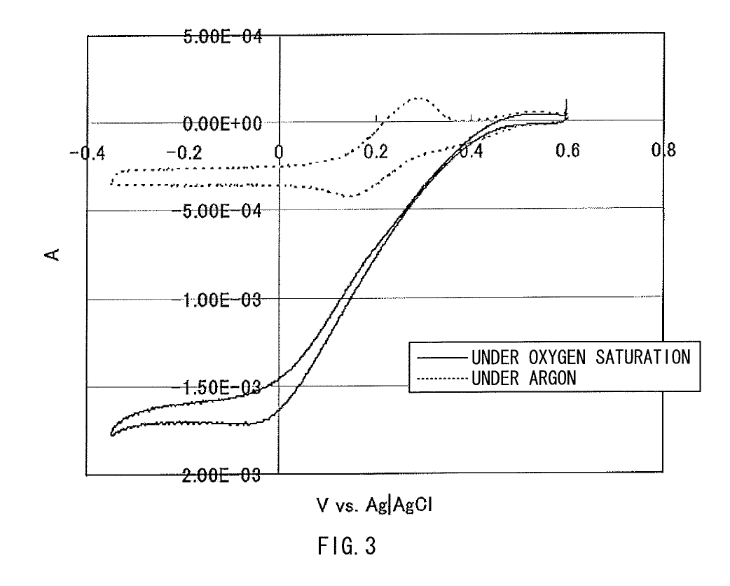

FIG. 3 is a voltammogram obtained in the electrode 1. In the diagram, a dotted line indicates voltammogram measured under argon, and the solid line indicates voltammogram measured under oxygen saturation. The difference between a current value under argon in which the catalyst reaction of BO does not proceed and a current value under oxygen saturation in which the catalyst reaction proceeds was set as a catalyst current value, and the difference of steady current values at −0.2V (vs. Ag|AgCl) was set to a catalyst current value for evaluation of the electrode performance.

Table 15 shows catalyst current values (mA/cm²) obtained in the electrodes 1 to 4.

TABLE 15

| | Catalyst current value | catalyst current |
|---|---|---|
| | (mA/cm2) | ratio (%) |
| Electrode 1 | 6.60 ± 0.57 | — |
| (commercial BO, existing | | |
| immobilizing method) | | |
| Electrode 2 | 2.96 ± 0.33 | 44.8% |
| (commercial BO, the | | |
| immobilizing method | | |
| according to the disclosure | | |
| Electrode 3 | 3.81 ± 0.22 | — |
| (heat-resistive BO variant, | | |
| existing immobilizing | | |
| method) | | |
| Electrode 4 | 4.56 ± 0.46 | 119% |
| (heat-resistive BO variant, | | |
| the immobilizing method | | |
| according to the disclosure | | |

The catalyst current value of the electrode 1 (commercial BO, existing immobilizing method) was 6.60±0.57 mA/cm². On the other hand, the catalyst current value in the electrode 3 to which the commercial BO was immobilized by the immobilizing method according to the disclosure was 2.06±0.33 mA/cm². The catalyst current ratio of the electrode 2 to the electrode 1 is 44.8%. It was confirmed that, in the case of using a commercial BO having low heat resistance, by setting the immobilization temperature to 60° C., the electrode performance deteriorates conspicuously.

The catalyst current value of the electrode 3 (heat-resistant BO variant, existing immobilizing method) was 3.81±0.22 mA/cm². On the other hand, the catalyst current value in the electrode 4 to which the heat-resistant BO variant was immobilized by the immobilizing method according to the disclosure was 4.56±0.46 mA/cm² and was increased to about 119% as compared with that of the electrode 3. It denotes that, in the case of using the heat-resistant BO variant, by setting the immobilization temperature to 60° C., the enzyme activity of BO increases and, therefore, the electrode having the electrode performance higher than that in the existing immobilizing method is obtained.

### INDUSTRIAL APPLICABILITY

The fuel cell, the electrode for the fuel cell, methods of manufacturing them, and the method of immobilizing enzyme to the electrode for the fuel cell according to the disclosure are widely applied to various devices, apparatuses, and systems necessitating electric power such as an electronic device, a mobile body, a power unit, a construction machine, a machine tool, a power generation system, and a cogeneration system.

It should be understood that various changes and modifications to the presently preferred example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1-13. (canceled)

14. A method for immobilizing enzyme on an electrode used for a fuel cell, wherein as the enzyme, an enzyme variant obtained by deletion, substitution, addition, or insertion of at least one amino acid residue in a wild-type amino acid sequence and having a characteristic that its activity increases through heat treatment is immobilized in a temperature range in which the activity can be increased.

15. The method for immobilizing enzyme of claim 14, wherein the electrode is a cathode, and the enzyme variant is variant bilirubin oxidase.

16. The method for immobilizing enzyme of claim 15, wherein the wild-type amino acid sequence is an amino acid sequence of bilirubin oxidase derived from an imperfect filamentous fungus, Myrothecium verrucari, represented by sequence number 1.

17. The method for immobilizing enzyme of claim 16, wherein as the variant bilirubin oxidase, a variant bilirubin oxidase represented by sequence number 2 in which phenylalanine at the 225th position from an N-terminal of the wild-type amino acid sequence is substituted with valine (F225V), asparagine acid at the 322nd position is substituted with asparagine (D322N), and methionine at the 468th position is substituted with valine (M468V) is used.

18. The method for immobilizing enzyme of claim 16, wherein as the variant bilirubin oxidase, a variant bilirubin oxidase represented by sequence number 3 in which phenylalanine at the 225th position from an N-terminal of the wild-type amino acid sequence is substituted with valine (F225V), asparagine acid at the 370th position is substituted with tyrosine (D370Y), and leucine at the 476th position is substituted with proline (L476P) is used.

19. The method for immobilizing enzyme of claim 16, wherein as the variant bilirubin oxidase, a variant bilirubin oxidase represented by sequence number 4 in which alanine at the 264th position from an N-terminal of the wild-type amino acid sequence is substituted with valine (A264V), alanine at the 418th position is substituted with threonine (A418T), and leucine at the 476th position is substituted with proline (L476P) is used.

20. The method for immobilizing enzyme of claim 16, wherein as the variant bilirubin oxidase, a variant bilirubin oxidase represented by sequence number 5 in which alanine at the 264th position from an N-terminal of the wild-type amino acid sequence is substituted with valine (A264V), arginine at the 437th position is substituted with histidine (R437H), and leucine at the 476th position is substituted with proline (L476P) is used.

21. The method for immobilizing enzyme of claim 16, wherein as the variant bilirubin oxidase, a variant bilirubin oxidase represented by sequence number 6 in which alanine at the 103rd position from an N-terminal of the wild-type amino acid sequence is substituted with proline (A103P), alanine at the 264th position from the N-terminal is substituted with valine (A264V), tyrosine at the 270th position is substituted with asparagine acid (Y270D), and leucine at the 476th position is substituted with proline (L476P) is used.

22. The method for immobilizing enzyme of claims 17, wherein the temperature range is from 20° C. to 65° C., both inclusive.

23. A method of manufacturing a fuel cell having a structure in which electrodes are opposed to each other with a proton conductor therebetween, comprising the step of immobilizing, on the electrode, an enzyme variant obtained by deletion, substitution, addition, or insertion of at least one amino acid residue in a wild-type amino acid sequence and having a characteristic that its activity increases through heat treatment in a temperature range in which the activity can be increased.

24. A fuel cell having a structure in which electrodes are opposed to each other with a proton conductor therebetween, wherein an enzyme variant obtained by deletion, substitution, addition, or insertion of at least one amino acid residue in a wild-type amino acid sequence and having a characteristic that its activity increases through heat treatment is immobilized on the electrode in a temperature range in which the activity can be increased.

25. A method of manufacturing an electrode for use in a fuel cell, comprising the step of immobilizing, on the electrode, an enzyme variant obtained by deletion, substitution, addition, or insertion of at least one amino acid residue in a wild-type amino acid sequence and having a characteristic that its activity increases through heat treatment in a temperature range in which the activity can be increased.

26. An electrode for use in a fuel cell, wherein an enzyme variant obtained by deletion, substitution, addition, or insertion of at least one amino acid residue in a wild-type amino acid sequence and having a characteristic that its activity increases through heat treatment is immobilized on the electrode in a temperature range in which the activity can be increased.


SEQUENCE LISTING (17 sequences)

SEQ ID NO: 1 - protein, 534 aa, Myrothecium verrucaria/NBRC(IFO)6113
VAQISPQYPMFTVPLPIPPVKQPRLTVTNPVNGQEIWYYEVEIKPFTHQVYPDLGSADLVGYDGMSPGPTFQVPRGVETVVRFINNAEAPNSVHLHGSFSRAAFDGWAEDITEPGSFKDYYYPNRQSARTLWYHDHAMHITAENAYRGQAGLYMLTDPAEDALNLPSGYGEFDIPMILTSKQYTANGNLVTTNGELNSFWGDVIHVNGQPWPFKNVEPRKYRFRFLDAAVSRSFGLYFADTDAIDTRLPFKVIASDSGLLEHPADTSLLYISMAERYEVVFDFSDYAGKTIELRNLGGSIGGIGTDTDYDNTDKVMRFVVADDTTQPDTSVVPANLRDVPFPSPTTNTPRQFRFGRTGPTWTINGVAFADVQNRLLANVPVGTVERWELINAGNGWTHPIHIHLVDFKVISRTSGNNARTVMPYESGLKDVVWLGRRETVVVEAHYAPFPGVYMFHCHNLIHEDHDMMAAFNATVLPDYGYNATVFVDPMEELWQARPYELGEFQAQSGQFSVQAVTERIQTMAEYRPYAAADE

SEQ ID NO: 2 - protein, 534 aa, Artificial
  Synthesized Bilirubin oxidase F225V/D322N/M468V variant
VAQISPQYPMFTVPLPIPPVKQPRLTVTNPVNGQEIWYYEVEIKPFTHQVYPDLGSADLVGYDGMSPGPTFQVPRGVETVVRFINNAEAPNSVHLHGSFSRAAFDGWAEDITEPGSFKDYYYPNRQSARTLWYHDHAMHITAENAYRGQAGLYMLTDPAEDALNLPSGYGEFDIPMILTSKQYTANGNLVTTNGELNSFWGDVIHVNGQPWPFKNVEPRKYRFRVLDAAVSRSFGLYFADTDAIDTRLPFKVIASDSGLLEHPADTSLLYISMAERYEVVFDFSDYAGKTIELRNLGGSIGGIGTDTDYDNTDKVMRFVVANDTTQPDTSVVPANLRDVPFPSPTTNTPRQFRFGRTGPTWTINGVAFADVQNRLLANVPVGTVERWELINAGNGWTHPIHIHLVDFKVISRTSGNNARTVMPYESGLKDVVWLGRRETVVVEAHYAPFPGVYMFHCHNLIHEDHDMVAAFNATVLPDYGYNATVFVDPMEELWQARPYELGEFQAQSGQFSVQAVTERIQTMAEYRPYAAADE

SEQ ID NO: 3 - protein, 534 aa, Artificial
  Synthesized Bilirubin oxidase F225V/D370Y/L476P variant
VAQISPQYPMFTVPLPIPPVKQPRLTVTNPVNGQEIWYYEVEIKPFTHQVYPDLGSADLVGYDGMSPGPTFQVPRGVETVVRFINNAEAPNSVHLHGSFSRAAFDGWAEDITEPGSFKDYYYPNRQSARTLWYHDHAMHITAENAYRGQAGLYMLTDPAEDALNLPSGYGEFDIPMILTSKQYTANGNLVTTNGELNSFWGDVIHVNGQPWPFKNVEPRKYRFRVLDAAVSRSFGLYFADTDAIDTRLPFKVIASDSGLLEHPADTSLLYISMAERYEVVFDFSDYAGKTIELRNLGGSIGGIGTDTDYDNTDKVMRFVVADDTTQPDTSVVPANLRDVPFPSPTTNTPRQFRFGRTGPTWTINGVAFAYVQNRLLANVPVGTVERWELINAGNGWTHPIHIHLVDFKVISRTSGNNARTVMPYESGLKDVVWLGRRETVVVEAHYAPFPGVYMFHCHNLIHEDHDMMAAFNATVPPDYGYNATVFVDPMEELWQARPYELGEFQAQSGQFSVQAVTERIQTMAEYRPYAAADE

SEQ ID NO: 4 - protein, 534 aa, Artificial
  Synthesized Bilirubin oxidase A264V/A418T/L476P variant
VAQISPQYPMFTVPLPIPPVKQPRLTVTNPVNGQEIWYYEVEIKPFTHQVYPDLGSADLVGYDGMSPGPTFQVPRGVETVVRFINNAEAPNSVHLHGSFSRAAFDGWAEDITEPGSFKDYYYPNRQSARTLWYHDHAMHITAENAYRGQAGLYMLTDPAEDALNLPSGYGEFDIPMILTSKQYTANGNLVTTNGELNSFWGDVIHVNGQPWPFKNVEPRKYRFRFLDAAVSRSFGLYFADTDAIDTRLPFKVIASDSGLLEHPVDTSLLYISMAERYEVVFDFSDYAGKTIELRNLGGSIGGIGTDTDYDNTDKVMRFVVADDTTQPDTSVVPANLRDVPFPSPTTNTPRQFRFGRTGPTWTINGVAFADVQNRLLANVPVGTVERWELINAGNGWTHPIHIHLVDFKVISRTSGNNTRTVMPYESGLKDVVWLGRRETVVVEAHYAPFPGVYMFHCHNLIHEDHDMMAAFNATVPPDYGYNATVFVDPMEELWQARPYELGEFQAQSGQFSVQAVTERIQTMAEYRPYAAADE

SEQ ID NO: 5 - protein, 534 aa, Artificial
  Synthesized Bilirubin oxidase A264V/R437H/L476P variant
VAQISPQYPMFTVPLPIPPVKQPRLTVTNPVNGQEIWYYEVEIKPFTHQVYPDLGSADLVGYDGMSPGPTFQVPRGVETVVRFINNAEAPNSVHLHGSFSRAAFDGWAEDITEPGSFKDYYYPNRQSARTLWYHDHAMHITAENAYRGQAGLYMLTDPAEDALNLPSGYGEFDIPMILTSKQYTANGNLVTTNGELNSFWGDVIHVNGQPWPFKNVEPRKYRFRFLDAAVSRSFGLYFADTDAIDTRLPFKVIASDSGLLEHPVDTSLLYISMAERYEVVFDFSDYAGKTIELRNLGGSIGGIGTDTDYDNTDKVMRFVVADDTTQPDTSVVPANLRDVPFPSPTTNTPRQFRFGRTGPTWTINGVAFADVQNRLLANVPVGTVERWELINAGNGWTHPIHIHLVDFKVISRTSGNNARTVMPYESGLKDVVWLGRHETVVVEAHYAPFPGVYMFHCHNLIHEDHDMMAAFNATVPPDYGYNATVFVDPMEELWQARPYELGEFQAQSGQFSVQAVTERIQTMAEYRPYAAADE

SEQ ID NO: 6 - protein, 534 aa, Artificial
  Synthesized Bilirubin oxidase A103P/A264V/Y270D/L476P variant
VAQISPQYPMFTVPLPIPPVKQPRLTVTNPVNGQEIWYYEVEIKPFTHQVYPDLGSADLVGYDGMSPGPTFQVPRGVETVVRFINNAEAPNSVHLHGSFSRAPFDGWAEDITEPGSFKDYYYPNRQSARTLWYHDHAMHITAENAYRGQAGLYMLTDPAEDALNLPSGYGEFDIPMILTSKQYTANGNLVTTNGELNSFWGDVIHVNGQPWPFKNVEPRKYRFRFLDAAVSRSFGLYFADTDAIDTRLPFKVIASDSGLLEHPVDTSLLDISMAERYEVVFDFSDYAGKTIELRNLGGSIGGIGTDTDYDNTDKVMRFVVADDTTQPDTSVVPANLRDVPFPSPTTNTPRQFRFGRTGPTWTINGVAFADVQNRLLANVPVGTVERWELINAGNGWTHPIHIHLVDFKVISRTSGNNARTVMPYESGLKDVVWLGRRETVVVEAHYAPFPGVYMFHCHNLIHEDHDMMAAFNATVPPDYGYNATVFVDPMEELWQARPYELGEFQAQSGQFSVQAVTERIQTMAEYRPYAAADE

SEQ ID NO: 7 - DNA, 1719 bp, Myrothecium verrucaria/NBRC(IFO)6113
atgttcaaacacacacttggagctgctgccctcagcttgcttttcaacagcaatgctgtccaggcaagccccgtccccgagacctcaccggcaactggacatctcttcaagcgagttgcccagatcagcccacagtatcccatgttcacagtaccactgccaattcctcctgttaagcagccccgcttgactgtaaccaatcctgtgaatggacaagagatctggtactatgaggtcgagatcaagcccttcactcaccaggtttaccctgaccttggatccgctgatctggtcgggtacgatggaatgtctcctggccctactttccaggttcctcgtggagttgaaacagttgtccgcttcattaacaatgctgaggctcctaactctgttcacttgcacggatcattctctcgtgccgcctttgacggatgggcagaggacatcaccgagcctggcagcttcaaagactattactacccaaacagacagtctgcccgtaccctatggtaccacgatcatgctatgcatatcactgctgagaacgcctaccgtggccaggctggtctctacatgctcactgacccagccgaagacgctctcaacttgccaagtggatatggcgagttcgatattccaatgatcctcacgtctaagcaatacaccgcaaacggcaacttggtcaccactaatggagagctgaactcattctggggtgatgtaattcacgtgaacggtcaaccctggcctttcaagaacgttgagcctcgcaaatatcgattccgcttcctcgatgccgcagtttctcgctctttcggcctttactttgctgatactgatgctatcgacactcgcttgcctttcaaggttattgcctccgattctggtcttcttgaacaccctgccgataccagcttgctgtacatttccatggccgagcgttacgaagttgtgtttgacttctccgactatgctggcaagactattgaactccgcaacctgggcggcagcattggcggcattggaacagataccgactatgataacaccgacaaggtcatgcgtttcgtggtagcagacgacacaactcagccagatacctcagttgttcctgctaaccttcgtgatgttcccttcccctctcccaccacaaacaccccccgacagttccgctttggtcgcaccggtcctacctggactattaatggtgttgcttttgctgatgttcaaaaccgtctgcttgcaaacgtacccgttggtactgtcgagcgttgggagctcatcaacgccggtaacggttggacgcaccctattcacatccatcttgtcgacttcaaggtcatttctcgtacttccggcaacaacgcgcgcacagtcatgccatacgagtccggtctcaaagacgttgtctggcttggtcgccgtgaaactgtggttgttgaggctcattacgcgcctttccctggtgtatacatgttccattgccacaatttgattcacgaggatcacgatatgatggctgcctttaacgccaccgtcctgccagattatggctataatgccactgttttcgttgaccctatggaagagctttggcaggctcgtccctatgaactcggcgagttccaggctcagagtggccagttcagcgttcaggctgttactgagcgtatccagactatggctgaatacagaccttacgccgcagctgacgagtag

SEQ ID NO: 8 - DNA, 34 bp, Artificial
  Synthesized Forward primer for BO cDNA amplification
gggaagcttatgttcaaacacacacttggagctg

SEQ ID NO: 9 - DNA, 34 bp, Artificial
  Synthesized Reverse primer for BO cDNA amplification
gggtctagactcgtcagctgcggcgtaaggtctg

SEQ ID NO: 10 - DNA, 18 bp, Artificial
  Synthesized Upstream sequence of the insert site of the plasmid vector
attaagaaatgttcaaac

SEQ ID NO: 11 - DNA, 19 bp, Artificial
  Synthesized Modified upstream sequence of the insert site of the plasmid vector
attaagaaaatgttcaaac

SEQ ID NO: 12 - DNA, 39 bp, Artificial
  Synthesized Forward primer for modification of upstream sequence of the insert site
ctatagggaatattaagaaaatgttcaaacacacacttg

SEQ ID NO: 13 - DNA, 41 bp, Artificial
  Synthesized Reverse primer for modification of upstream sequence of the insert site
caagtgtgtgtttgaacattttcttaatattccctatagtg

SEQ ID NO: 14 - DNA, 33 bp, Artificial
  Synthesized Forward primer for error-prone PCR
gtaaccaatcctgtgaatggacaagagatctgg

SEQ ID NO: 15 - DNA, 35 bp, Artificial
  Synthesized Reverse primer for error-prone PCR
gggataggcttaccttcgaagggccctctagactc

SEQ ID NO: 16 - DNA, 32 bp, Artificial
  Synthesized Forward primer for amplification of the inserted BO gene
gggaattcttgcccagatcagcccacagtatc

SEQ ID NO: 17 - DNA, 32 bp, Artificial
  Synthesized Reverse primer for amplification of the inserted BO gene
gggactagtcactcgtcagctgcggcgtaagg